(19) United States
(12) Patent Application Publication
(10) Pub. No.: US 2022/0087298 A1
(43) Pub. Date: Mar. 24, 2022

(54) METHOD FOR PREPARING GOS-PREPARATION WITH BETA-GALACTOSIDASE FROM CRYPTOCOCCUS TERRESTRIS, GOS PREPARATIONS OBTAINABLE THEREBY AND USES THEREOF

(71) Applicant: FrieslandCampina Nederland B.V., Amersfoort (NL)

(72) Inventors: Mirre VISKAAL-VAN DONGEN, Wageningen (NL); Linqiu CAO, Wageningen (NL)

(21) Appl. No.: 17/420,062

(22) PCT Filed: Nov. 27, 2019
(86) PCT No.: PCT/EP2019/082732

§ 371 Date: Jun. 30, 2021

(30) Foreign Application Priority Data
Jan. 2, 2019 (EP) 19150029.7

(51) Int. Cl. A23L 33/21; A61K 31/702

(52) U.S. Cl. CPC A23L 33/21; A61K 31/702

(57) ABSTRACT

The invention relates to the field of nutritional ingredients, in particular to economically attractive methods for producing hypoallergenic galactooligosaccharides (HA-GOS) and the use thereof in food and feed items. Provided is a method for the production of a HA-GOS preparation, comprising contacting a lactose feed with a beta-galactosidase (EC 3.2.1.23) comprising an amino acid sequence according to any of SEQ ID NO: 1, 2, 3 or 4, or an amino acid sequence that is at least 80% identical thereto, wherein the lactose feed is a cheese whey permeate (CWP) or a CWP that is enriched in sialyllactose (SL-CWP).

The invention relates to the field of nutritional ingredients. More in particular, it relates to economically attractive methods for producing hypoallergenic galacto-oligosaccharides and the use thereof in food and feed items. The invention particularly relates to the use of cheese whey permeates as lactose feed in a transgalactosylation reaction catalyzed by a beta-galactosidase derived from Cryptococcus terrestris in the production of a hypoallergenic GOS preparation.

The term “GOS” stands for galacto-oligosaccharides (GOS), which generally comprise a chain of galactose units and a terminal glucose unit, that arise through consecutive transgalactosylation reactions (trans glycosylation reactions), catalyzed by a beta-galactosidase. Some of the GOS components exist naturally in human breast milk and bovine colostrum. Typical GOS preparations mainly comprise di- to hexa-saccharides.

Various physiological functions of GOS have been reported, including the capacity to stimulate the growth of bifidogenic bacteria in the gut, to support normal gut transit, to contribute to natural defenses and to enhance mineral absorption. GOS has received particular attention for their prebiotic effects that promote the growth of Bifidobacterium, Lactobacillus, and other enteric bacteria. Therefore, GOS is commonly used in infant formula, beverages fermented by Lactobacillus, and yogurts. Some of these foods containing GOS are certified as Food for Specified Health Uses by the Consumer Affairs Agency in Japan, and GOS is certified as generally recognized as safe (GRAS) substances by the U.S. Food and Drug Administration (GRAS Notices: GRN 233, 236, 285, 286, 334, 484, 489, 495, 518, and 569).

In general, GOS is produced by a transglycosylation reaction with a beta-galactosidase enzyme (enzyme class EC.3.2.1.23). beta-Galactosidase enzymes are produced in many microorganisms such as Bacillus circulans, Aspergillus oryzae, Kluyveromyces marxianus, Kluyveromyces fragilis, Sporobolomyces singularis, and Lactobacillus fermentum. Beta-galactosidases differ in their three-dimensional structures, resulting in stereo- and regioselectivity of the glycosidic bonds that are formed. For example, typically a fungal beta-galactosidase derived from Aspergillus predominantly produces ß1-6 bonds (thus resulting in a GOS preparation that predominantly comprises ß1-6 bonds, which may be referred to as “6′-GOS”), while a bacterial beta-galactosidase derived from Bacillus predominantly produce ß1-4 bonds (resulting in a GOS preparation that predominantly comprises ß1-4 bonds, which may also be referred to as “4′-GOS”). Moreover, beta-galactosidase produced by B. circulans possesses particularly strong transgalactosylation activity, and thus, GOS prepared by B. circulans beta-galactosidase is sold worldwide. Since introduction to the market (1999), approximately more than 100 million of infants have consumed infant formula containing GOS prepared by beta-galactosidase from B. circulans. It has been proven to be a safe ingredient, with a GRAS status acknowledged by the FDA. In the past few years, however, a small number of very rare cases of GOS-related allergy has been reported in South East Asia. Research has shown that certain oligosaccharide structures present in GOS can exert an allergic response in very sensitive subjects.

Jyo et al. (Occup. Environ. Allergy 3, 12-20 (1992)) determined allergy symptoms after consumption of a lactobacillus beverage containing 6′-GOS produced by fungal beta-galactosidase. Allergy symptoms have also been observed with 4′-GOS produced by bacterial beta-galactosidase. In 2014, Kaneko et al. (Biosc. Biotechnol. Biochem. 78, 100-108) observed that GOS produced by treating lactose with a beta-galactosidase derived from B. circulans may induce allergic reactions and revealed that the allergies were caused by two tetrasaccharides [Gal ß1-4 (Gal ß1-4 Gal ß1-6) Glc, Gal ß1-4 Gal ß1-4 Gal ß1-3 Glc]. These GOS allergy cases occurred in subjects who already had a history of atopy.

WO 2017/115826 by Amano discloses the screening of various kinds of microorganisms for novel ß-galactosidase enzymes having desirable properties for industrial applications from viewpoints of heat resistance, pH stability, and others. This resulted in the identification of enzymes of a microorganism (wild-type strain) of the genus Cryptococcus, in particular Cryptococcus terrestris (recently renamed Papiliotrema terrestris).

The high costs of purified lactose makes conventional processes for GOS production less attractive for large scale industrial application, in particular to make GOS available as a cost-effective ingredient in a broad range of food and feed applications, for example for the growing up milk (GUM) market.

The present inventors therefore aimed at developing a process for the manufacture of a GOS preparation that has a higher economic feasibility, while at least preserving the desirable traits such as bifidogenic properties of the GOS product.

It was surprisingly observed that when cheese whey permeate (CWP), a side-product of the cheese making process, was used as a source of lactose in a method for preparing GOS using a C. terrestris ß-galactosidase or a mutant thereof, a GOS preparation having an unexpected low allergenicity was obtained. Such hypoallergenic GOS preparation is herein referred to as “HA-GOS”. Moreover, it was found that a lactose-depleted whey permeate, abbreviated to DLP or OPL, has a high sialyllactose (SL) content and is therefore highly suitable to prepare a low-cost, SL-enriched HA-GOS preparation. Such CWP enriched in SL is herein also referred to as SL-CWP. SL is a component of human milk oligosaccharides known to have significant health benefits because of its role in supporting resistance to pathogens, gut maturation, immune function, and cognitive development.

Since CWP and OPL represent the most abundant dairy waste streams, the invention herewith not only provides an economically attractive procedure to produce HA-GOS having higher SL levels compared to regular GOS preparations, but it also allows the valorization of an industrial waste stream into HA-GOS.

The invention accordingly relates to a method for the production of a galacto-oligosaccharide (HA-GOS) preparation, comprising contacting a lactose feed with a beta-galactosidase (EC 3.2.1.23) comprising an amino acid sequence according to any of SEQ ID NO: 1, 2, 3 or 4, or an amino acid sequence that is at least 80% identical thereto (meaning: at least 80% identical to SEQ ID NO: 1, at least 80% identical to SEQ ID NO: 2, at least 80% identical to SEQ ID NO: 3, and/or at least 80% identical to SEQ ID NO: 4), wherein the lactose feed comprises a cheese whey permeate (CWP) or a CWP that is enriched in sialyllactose (SL-CWP).

The invention also relates to a GOS preparation obtainable by a method according to the invention.

The invention further relates to the use of the GOS preparation in a method of at least partially preventing an (IgE-mediated) allergic response in a subject.

The invention also provides a method for at least partially preventing hypersensitivity to a GOS preparation in a subject, comprising administering a (hypoallergenic) nutritional composition comprising the GOS preparation according to the invention.

The invention further relates to a method for providing a hypoallergenic nutritional composition, comprising (i) providing the GOS preparation according to a method of the invention, and (ii) formulating said GOS preparation together with at least one further hypoallergenic or non-allergenic ingredient into a hypoallergenic nutritional composition. Also provided is a hypoallergenic nutritional composition obtainable by such method.

The invention also relates to a nutritional composition comprising the HA-GOS preparation according to the invention.

### Lactose Feed

The method of the invention is among others characterized in the use of a raw material derived from cow's milk as lactose feed. The raw material comprises a cheese whey permeate (CWP) or a CWP that has been processed further to remove unwanted components and/or to enrich for desirable components. CWP is a lactose-rich effluent remaining after protein extraction from milk-resulting cheese whey, an abundant dairy waste. In all milk-producing countries, milk is primarily used to manufacture cheese. However, only approximately half of the solids present in milk are coagulated and recovered as cheese; the remaining half are recovered as whey, a by-product of the cheese manufacturing process. Whey contains mainly proteins, lactose, minerals and vitamins. The treatment and disposal of whey frequently poses significant environmental problems. Whey is sometimes dried to be used as an extender in cattle feed, or in other processed food items such as candy bars. However, the use in food applications is limited because of the high ash content of whey. Using more advanced techniques of ultrafiltration, commercially valuable proteins can be recovered from whey. Whey proteins have long been used as food ingredients and in pharmaceutical applications. In ultrafiltration, pressure is applied to a solution to force whey through a semipermeable membrane. The openings of the membrane are sized to pass all portions of the whey except the proteins, which become concentrated. The material which passes through the membrane is called (cheese) whey permeate, also known as liquid permeate. Whey permeate contains mainly lactose, minerals and vitamins.

In one embodiment of the invention, the whey permeate is used as such, i.e. without further treatment, as lactose feed in a transgalactosidation reaction. It is however preferred that the permeate is demineralized prior to contacting with the beta-galactosidase. In one embodiment, the lactose feed is a CWP that is demineralized to an ash content of up to about 4 wt. %, or a conductivity of up to about 4 mS. Alternatively, the demineralization step can be carried out after the conversion to GOS. Demineralization may be performed by methods known in the art, including electrodialysis (ED), reverse osmosis (RO), nanofiltration (NF) or ion exchange technology. In one preferred aspect, the lactose feed is CWP that has been subjected to electrodialysis (CWP-ED). In a further preferred embodiment, the lactose feed is CWP that has been subjected to electrodialysis to an ash content of up to 4 wt. % and/or a conductivity of up to 4 mS.

An exemplary process for the preparation of HA-GOS with CWP as lactose feed comprises the steps of:
1. Concentrating CWP to a dry matter content of >25%, preferably to a dry matter content of about 28 wt. % or more;
2. Demineralization, e.g. by ED, preferably to an ash content of less than or equal to 4 wt. % and/or a conductivity of up to 4 mS;
3. Concentrating the demineralized CWP, preferably to a dry matter content of 50 wt. % or more, preferably 55 wt. % or more;
4. Adding enzyme (a beta-galactosidase derived from Cryptococcus terrestris) to convert lactose to HA-GOS;
5. Deactivating the enzyme (e.g. by a heat treatment as is known to the skilled person);
6. Concentrating the HA-GOS solution, preferably to a dry matter content of 70% or more, to obtain a HA-GOS syrup.

Alternatively, the demineralization step may also be performed after, instead of before, the conversion of lactose to HA-GOS.

The HA-GOS solution obtained in step 5 may also undergo additional purification steps, e.g. to remove mono-sugars and/or remaining lactose. The HA-GOS solution, preferably after at least removal of mono-sugars, may further be dried in order to obtain HA-GOS in powder form.

In another preferred aspect, the lactose feed is CWP has been subjected to a treatment to enrich for sialyllactose (SL), herein also referred to as SL-CWP. For example, SL-CWP can be obtained by a process commonly used in the dairy industry to obtain lactose from whey permeate by evaporating and crystallizing. Alternatively the lactose removal step may comprise spray-drying the concentrated material and then adding water to dissolve the oligosaccharides whilst leaving the lactose in a crystallised form.

The recovery of lactose from whey permeate results in the by-product delactosed whey permeate (DLP or OPL), which heretofore was considered to contain unrecoverable lactose and protein, and the minerals and vitamins originally present in the whey permeate. However, with the lactose removal the levels of sialic acid containing compounds increased, including the content of sialyllactose. This by-product is difficult to handle, and is typically land-spread (which leads to runoff, in some countries resulting in serious pollution problems in lakes and rivers), landfilled or sold for cattle feed at a loss to the factory.

Interestingly however, OPL contains not only more than 50% lactose but also 0.3-0.4% sialyl-lactose (2,3-sialyllactose and 2,6-sialyl lactose) and possibly other valuable bovine milk oligosaccharides (bMOs) as well. Besides, it has been shown that bMOs contain as many oligosaccharides as found in human oligosaccharides (hMOs). Therefore, combining of bMOs with GOS can provide a unique GOS product that may have the complete features of GOS but partially the features of hMOs or bMOs (for details see Table 1).

As illustrated in FIG. 2, lactose in OPL can be converted into normal GOS while sialyl-lactose (such as 2,3-sialyllactose and 2,6-sialyl-lactose) will remain intact.

The present finding that delactosed whey permeate is advantageously converted to a valuable food or feed ingredient opens up a new way for the valorization of this large volume by-product of the dairy industry.

In one embodiment, the lactose feed is a SL-CWP having a sialyllactose content of at least 0.25 wt. %, preferably at least 0.3 wt. % based on dry solids.

The lactose-content of the SL-CWP can vary, but it is typically in the range of about 52-60 wt. %.

It is known that OPL contains also around 24% monovalent salt and 8% divalent salts, 8% NPN and a few percent of proteins. For example, a typical OPL stream contains roughly 24% Ash, 7% NPN, 7.8% protein, 57% lactose and 0.3-0.4% SL (see Table 2). Especially the high salt concentration may interfere with the enzymatic reaction. Accordingly, a method of the invention advantageously comprises the use of SL-CWP that is produced from an (industrially produced) OPL solution that has been subjected to a demineralization step, in particular a treatment to remove anions.

TABLE 2
Typical composition of OPL.

| | | gram/kg OPL |
|---|---|---|
| Ingredient | % | (dry matter) |
| Na | 2.1 | 21 |
| K | 7.8 | 78 |
| Ca | 1 | 10 |
| Mg | 0.25 | 2.5 |
| PO4— | 5.8 | 58.0 |
| citrate | 5.8 | 58 |
| Cl— | 7 | 70 |
| NPN | 7.1 | |
| lactose | 57 | |
| Protein | 7.8 | |
| Ash | 23.7 | |
| SL total | 0.3 | |

Remarkably, OPL contains relatively low amounts of calcium. Therefore, in order to remove multivalent anions such as PO₄³⁻, citrate³⁻, extra calcium can be added to enhance the formation of insoluble calcium monohydrogen phosphate and calcium citrate, thus allowing for easy removal of salts by e.g. centrifugation. In a preferred embodiment, OPL is treated with lime (CaO/Ca(OH)₂) to precipitate anions.

The lactose feed may totally consist of (demineralized) CWP or SL-CWP. However, it is also possible to replace part of the (demineralized) CWP or SL-CWP with lactose, which lactose does not have to be extremely pure, such as edible lactose or lactose slurry. If part of the (demineralized) CWP or SL-CWP is replaced by lactose, the lactose feed is preferably made up from at least 20 wt %, more preferably at least 30 wt % (demineralized) CWP or SL-CWP.

The invention herewith also provides a method for the production of HA-GOS, comprising contacting a lactose feed with a beta-galactosidase (EC 3.2.1.23) comprising an amino acid sequence according to any of SEQ ID NO: 1, 2, 3 or 4, or an amino acid sequence that is at least 80% identical thereto (meaning: at least 80% identical to SEQ ID NO: 1, at least 80% identical to SEQ ID NO: 2, at least 80% identical to SEQ ID NO: 3, and/or at least 80% identical to SEQ ID NO: 4), wherein the lactose feed comprises a CWP that has been subjected to a lactose-removal step, preferably followed by a step to remove multivalent anions.

For example, FIG. 3 depicts a process chart of an exemplary method comprising the following main steps:
1. Addition of lime (CaOH) to OPL concentrated solution (˜37% dry matter) to form insoluble calcium salts such as calcium phosphate and calcium citrate;
2. Centrifugation to remove the insoluble salts;
3. Adding enzyme (a beta-galactosidase derived from Cryptococcus terrestris) to convert lactose to HA-GOS;
4. Deactivating the enzyme (e.g. by a heat treatment as is known to the skilled person);
5. Electrodialysis (ED) to remove monovalent salts;
6. Nanofiltration (NF) aiming to remove the mono-sugars, resulting in a HA-GOS composition with more than 70% purity;
7. Optionally concentrating the HA-GOS syrup, e.g. by NF, preferably to a dry matter content of 70% or more, more preferably to a dry matter content of 75% or more.

The HA-GOS solution obtained in step 6 may be dried, in order to obtain HA-GOS in powder form.

The typical dry matter content of a concentrated OPL stream is around 37%, thus containing a low total lactose concentration. Consequently, a direct enzymatic conversion of this concentrated OPL stream may lead to a low efficiency in lactose to GOS conversion. This drawback associated with OPL valorization can be overcome by first converting OPL into OPL-GOS, followed by a consecutive removal of the salts and/or mono sugars by ED and NF It is preferred to perform these separation steps anyway when a high purity HA-GOS preparation is desired. However, the multivalent anions can still be removed before the ED and NF steps.

### Transgalactosylation Reaction by Beta-Galactosidase

In general, a ß-galactosidase shows a lactose hydrolyzing activity (an activity to hydrolyze lactose by the action on the ß-1,4 bond) and a transgalactosylation activity (an activity to transfer galactose). Therefore, the expression “ß-galactosidase activity” as used herein is intended to include such two activities.

The enzyme for use according to the invention has been previously described in WO 2017/115826. It is a ß-galactosidase derived from the yeast Cryptococcus terrestris. Herein, by “ß-galactosidase derived from C. terrestris” is meant a ß-galactosidase enzyme produced by a microorganism (of either a wild-type strain or a mutant strain) which is classified into Cryptococcus terrestris, or a ß-galactosidase enzyme obtained by genetic engineering procedures using the ß-galactosidase gene from a microorganism (of either a wild-type strain or a mutant strain) which is classified into Cryptococcus terrestris. Therefore, the term “ß-galactosidase derived from Cryptococcus terrestris” also encompasses a recombinant enzyme that is produced by a host microorganism into which the ß-galactosidase gene (or a modified gene thereof) obtained from Cryptococcus terrestris has been introduced. Cryptococcus terrestris was recently renamed Papiliotrema terrestris.

WO 2017/115826 describes three kinds of ß-galactosidase produced by mutant strains derived from the Cryptococcus microorganism (mutant strain enzymes 1, 2, and 3, respectively), and discloses their amino acid sequences. These three ß-galactosidase enzymes were found to have a partial sequence of the full-length amino acid sequence of the wild-type strain enzyme (the wild-type strain enzyme is SEQ ID NO: 1), which is deduced from its gene sequence. Specifically, these mutant enzymes are one having an amino acid sequence in which the N-terminal 130 amino acid residues of the full-length amino acid sequence of the wild-type strain enzyme are deleted, which is referred to as “mutant strain enzyme 1” for the purpose of description (SEQ ID NO: 2); one having an amino acid sequence in which the N-terminal 136 amino acid residues of the full-length amino acid sequence of the wild-type strain enzyme are deleted (SEQ ID NO:3), which is referred to as “mutant strain enzyme 2” for the purpose of description; and one having an amino acid sequence in which the N-terminal 141 amino acid residues of the full-length amino acid sequence of the wild-type strain enzyme are deleted (SEQ ID NO:4), which is referred to as “mutant strain enzyme 3”.

Accordingly, a ß-galactosidase enzyme for use in a method of the present invention comprises the amino acid sequence of any one of SEQ ID NOs: 1, 2, 3 or 4, or an equivalent amino acid sequence showing at least 80% sequence identity thereto. These enzymes are herein also referred to as “Tetris enzymes”. Also, a combination of two or more ß-galactosidase enzymes may be used, wherein each enzyme comprises an amino acid sequence of any one of SEQ ID NOs: 1, 2, 3 or 4 or an amino acid sequence equivalent to any one of said amino acid sequences. In one embodiment, at least one mutant enzyme (SEQ ID NO's 2, 3 or 4, or an equivalent thereof) is used. Preferably two or more mutant enzymes are used, optionally in combination with the wild type enzyme. For example, a combination of two or more enzymes, each enzyme comprising an amino acid sequence of SEQ ID NO: 2, SEQ ID NO: 3 and SEQ ID NO: 4, or an amino acid sequence equivalent to said amino acid sequences, is used. In a specific aspect, the enzyme combination comprises a mixture of the three distinct mutant enzymes and the wildtype enzyme.

The term “equivalent amino acid sequence” in this case means an amino acid sequence which is partially different from the reference amino acid sequence (i.e. amino acid sequence of any one of SEQ ID NOs:1 to 4), but the difference does not substantially influence the function of the protein (beta-galactosidase activity). Thus, an enzyme having a polypeptide chain of the equivalent amino acid sequence shows a beta-galactosidase activity.

The degree of the activity is not particularly limited as long as the function of a beta-galactosidase can be exhibited, but is preferably equivalent to or higher than that of the enzyme having a polypeptide chain of the reference sequence. Preferably, the length of the equivalent amino acid sequence is not longer than that of the sequence of SEQ ID NO: 1. The term “partial difference in the amino acid sequence” typically means mutation (change) in the amino acid sequence caused by deletion or substitution of one to several (up to, for example, 3, 5, 7, or 10) amino acids composing the amino acid sequence, or addition, insertion, or a combination thereof of one to several (up to, for example, 3, 5, 7, or 10) amino acids. The difference in the amino acid sequence is acceptable as long as the beta-galactosidase activity is maintained (the activity may be varied to a degree). As long as the conditions are satisfied, the position of the difference in the amino acid sequence is not particularly limited, and the difference may arise in a plurality of positions. The term “plurality” means, for example, a number corresponding to less than about 20%, preferably less than about 15%, more preferably less than about 10%, even more preferably less than about 5% of the total amino acids, and most preferably less than about 1%. More specifically, the equivalent protein has about 80% or more, preferably about 85% or more, more preferably about 90% or more, much more preferably about 95% or more, even more preferably about 97% or more, and most preferably about 99% or more identity with the reference amino acid sequence.

The difference of the amino acid sequence may arise in a plurality of positions. Preferably, the equivalence protein is obtained by causing conservative amino acid substitution in an amino acid residue which is not essential for beta-galactosidase activity. The term “conservative amino acid substitution” means the substitution of an amino acid residue with another amino acid residue having a side chain with similar properties.

Amino acid residues are classified into several families according to their side chains, such as basic side chains (for example, lysine, arginine, and histidine), acidic side chains (for example, aspartic acid and glutamic acid), uncharged polar side chains (for example, glycine, asparagine, glutamine, serine, threonine, tyrosine, and cysteine), nonpolar side chains (for example, alanine, valine, leucine, isoleucine, proline, phenylalanine, methionine, and tryptophan), ß-branched side chains (for example, threonine, valine, and isoleucine), and aromatic side chains (for example, tyrosine, phenylalanine, tryptophan, and histidine). Conservative amino acid substitution is preferably the substitution between amino acid residues in one family.

The identity (%) between two amino acid sequences or two nucleic acid sequences (hereinafter, the term “two sequences” are used for representing either of two sequences) can be determined by the following procedure. Firstly, two sequences are aligned for optimum comparison of the two sequences (for example, a gap may be introduced into the first sequence so as to optimize the alignment with respect to the second sequence). When a molecule (amino acid residue or nucleotide) at a specific position in the first sequence and a molecule in the corresponding position in the second sequence are the same as each other, the molecules in the positions are defined as being identical. The identity between two sequences is a function of the number of identical positions shared by the two sequences (i.e., identity (%)=number of identical positions/total number of positions %100). Preferably, the number and size of the gaps, which are required to optimize the alignment of the two sequences, are taken into consideration.

The comparison and determination of the identity between two sequences can be carried out by using a mathematical algorithm. A specific example of the mathematical algorithm that can be used for comparing the sequences includes an algorithm described in Karlin and Altschul (1990) Proc. Natl. Acad. Sci. USA 87:2264-68 and modified by Karlin and Altschul (1993) Proc. Natl. Acad. Sci. USA 90:5873-77. However, the algorithm is not necessarily limited to this. Such an algorithm is incorporated in NBLAST program and XBLAST program (version 2.0) described in Altschul et al. (1990) J. Mol. Biol. 215: 403-10. In order to obtain an equivalent nucleic acid sequence, for example, BLAST nucleotide search with score=100 and word length=12 may be carried out by the NBLAST program. In order to obtain an equivalent amino acid sequence, for example, BLAST polypeptide search with score=50 and word length=3 may be carried out by the XBLAST program. In order to obtain gapped alignments for comparison, Gapped BLAST described in Altschul et al., (1997) Amino Acids Research 25(17): 3389-3402 can be utilized. In using BLAST and Gapped BLAST, the default parameters of the corresponding programs (e.g., XBLAST and NBLAST) can be used. In detail, see http://www.ncbi.nlm.nih.gov. Another example of the mathematical algorithm that can be used for comparing sequences includes an algorithm described in Meyers and Miller (1988) Comput. Appl. Biosci. 4: 11-17. Such programs are incorporated into the ALIGN program that can be used for, for example, GENESTREAM network server (IGH Montpellier, France) or ISREC server. When the ALIGN program is used for comparison of the amino acid sequences, for example, PAM120 weight residue table can be used in which a gap length penalty is 12 and a gap penalty is 4.

The identity between two amino acid sequences can be determined by using the GAP program in the GCG software package, using Blossom 62 matrix or PAM250 matrix with the gap weight of 12, 10, 8, 6, or 4, and the gap length weight of 2, 3, or 4. The identity between two nucleic acid sequences can be determined by using the GAP program in the GCG software package (available at http://www.gcg.com), with the gap weight of 50, and the gap length weight of 3. The enzyme may be a portion of a larger protein (for example, a fusion protein). Examples of the sequence added to a fused protein include the sequences/tags useful for purification of multiple histidine residues, and addition sequences which ensures stability in recombinant production.

As demonstrated in the Examples section of WO 2017/115826, 6-galactosidase enzymes were isolated and purified from Cryptococcus terrestris strain MM13-F2171 and its mutant strains M2 and M6. Mutant strains (M2 and M6) were obtained from Cryptococcus terrestris strain MM13-F2171 by means of mutagenesis with UV treatment. Cryptococcus terrestris strains MM13-F2171 and M2 have been deposited at a depository, as described below, and are readily available.

<Cryptococcus terrestris Strain MM13-F2171>
Depository: Patent Microorganisms Depositary, National Institute of Technology and Evaluation (Room 122, 2-5-8 Kazusa Kamatari, Kisarazu-shi, Chiba, 292-0818, JAPAN). Identification reference: Cryptococcus terrestris MM13-F2171. Date of deposit: Dec. 10, 2015. Accession number: NITE BP-02177;
   <Cryptococcus terrestris Strain M2>
Depository: Patent Microorganisms Depositary, National Institute of Technology and Evaluation (Room 122, 2-5-8 Kazusa Kamatari, Kisarazu-shi, Chiba, 292-0818, JAPAN). Identification reference: Cryptococcus terrestris APC-6431. Date of deposit: Dec. 10, 2015. Accession number: NITE BP-02178

Accordingly, in one embodiment the enzyme used in the present invention is the enzyme isolated from Cryptococcus terrestris strain MM13-F2171 (Accession Number: NITE BP-02177) or APC-6431 (Accession Number: NITE BP-02178).

The reaction conditions for transgalactosylation can be determined by a person skilled in the art. Relevant criteria include reaction temperature, reaction time, initial lactose content of lactose feed, pH, enzyme dosage, and the like. As is shown herein below, it was found that good results were obtained with CWP or demineralized CWP as lactose feed when the lactose feed was contacted with beta-galactosidase at a temperature of about 60-75° C., preferably about 63-73° C., more preferably about 65-70° C., at a lactose content in the range of 45-58 wt. % lactose, preferably 47-55 wt. % lactose, and/or an enzyme dosage of 0.60-1.1 LU/gram, preferably of 0.65-1.0 LU/gram. In one specific aspect, the reaction time is 10-30 hours, preferably 20-26 hours, the enzyme dosage is at least 0.60 LU/gram lactose, the lactose concentration is >50% and the temperature is about 60-70° C. In one specific aspect, the reaction time is 10-30 hours, preferably 20-26 hours, the enzyme dosage is at least 0.60 LU/gram lactose, the lactose concentration is >50% and the temperature is in the range of 65-70° C. In another specific aspect, the reaction time is 10-30 hours, preferably 20-26 hours, the enzyme dosage is at least 0.65 LU/gram lactose, the lactose concentration is >50% and the temperature is about 70° C.

In another specific aspect, the reaction time is at least 36 hours, like 40-48 hours, the enzyme dosage is 0.65-0.75 LU/gram, the reaction temperature is in the range of 60-75° C., preferably in the range of 63-73° C., more preferably in the range of 65-70° C., and the lactose concentration about 50%.

In another specific aspect, the reaction time is at least 36 hours, like 40-48 hours, the enzyme dosage is 0.75 LU/gram, the reaction temperature is about 65° C. and the lactose concentration about 50%.

Experiments performed using a SL-CWP lactose feed indicated that SL-CWP is advantageously contacted with beta-galactosidase at a temperature of about 55-75° C., preferably about 60-70° C., more preferably about 65-70° C. at pH 6.0-7.0 and/or at an enzyme dosage of 0.60-2.5 LU/gram, preferably 0.65-2.0 LU/gram.

In one embodiment, the invention provides a method for the production of a HA-GOS preparation, comprising contacting a beta-galactosidase (EC 3.2.1.23) comprising an amino acid sequence according to any of SEQ ID NO: 1, 2, 3 or 4, or an amino acid sequence that is at least 80% identical thereto (meaning: at least 80% identical to SEQ ID NO: 1, at least 80% identical to SEQ ID NO: 2, at least 80% identical to SEQ ID NO: 3, and/or at least 80% identical to SEQ ID NO: 4), at an enzyme dosage of 0.60-1.0 LU/gram, preferably 0.75-1.0 LU/gram with a CWP that has been subjected to a lactose-removal step followed by a step to remove divalent anions, at an initial lactose content of at least 60 wt. %. For example, the reaction is performed at 65° C. using 65% (w/w) OPL at an enzyme dosage of 2 LU/gram lactose. After 24 hour reaction, the GOS formed can be obtained by centrifugation and the enzyme may be denatured, e.g. by heating at 100° C. for 15 minutes.

In a method according to the invention, the HA-GOS thus obtained may be subjected to at least one further purification step, preferably wherein said purification step comprises one or more of demineralization, de-proteinization, removal of mono-sugar components, enriching for sialyllactose or removal of coloured impurities (e.g. by treatment with activated carbon). In one embodiment, HA-GOS is subjected to a nanofiltration (NF) or ultrafiltration step (UF) to remove mono-sugars and protein. A further concentration step, for example using NF or evaporation, may be performed to obtain a concentrated, preferably SL-enriched, HA-GOS preparation having a dry matter content of at least 70%, preferably at least 75%.

### HA-GOS

Also provided herein is a hypoallergenic GOS preparation obtainable by a method according to the invention.

As used herein, the term “hypoallergenic GOS preparation” (abbreviated to HA-GOS) refers to a GOS composition not triggering Bc-GOS induced allergy. In other words, the term “hypoallergenic GOS preparation” refers to a GOS composition that, when administered to a subject suffering from an allergy caused by GOS produced by Bacillus circulans beta-galactosidase, evokes a reduced allergic response when compared to a GOS preparation produced by Bacillus circulans beta-galactosidase. More in particular, a hypoallergenic GOS preparation has a decreased score in a Basophil Activation Test performed on a blood sample isolated from the subject when compared to a GOS preparation obtained by Bacillus circulans.

In a specific aspect, the invention provides a hypoallergenic GOS preparation comprising at least 0.3 wt. % of sialyllactose, preferably at least 0.35 wt. % sialyllactose. This HA-GOS preparation can be obtained when using SL-CWP as lactose feed. For example, provided herein is a HA-GOS preparation comprising at least 50 wt. % of DP2-DP7 GOS (ensuring a bifidogenic effect) and at least 0.30 wt. % sialyllactose, based on total dry matter. Removal of lactose and mono-sugars from such preparation provided a HA-GOS preparation comprising at least 70 wt. %, preferably at least 75 wt. %, of DP2-DP7 GOS (ensuring a bifidogenic effect) and at least 0.35 wt. % sialyllactose, based on total dry matter. The HA-GOS preparation has a GOS composition similar that of a GOS preparation that is prepared from purified lactose feed, but is enriched in sialyllactose. In addition, vitamins that were present in the SL-CWP lactose feed may be present in the HA-GOS preparation. Also (part of) the minerals present in the SL-CWP lactose feed may be present.

Also provided is a HA-GOS preparation obtained when using CWP as lactose feed. Such a HA-GOS preparation comprises at least 50 wt. % of DP2-DP7 GOS based on total dry matter. Removal of lactose and mono-sugars from such preparation provided a HA-GOS preparation comprising at least 70 wt. %, preferably at least 75 wt. %, of DP2-DP7 GOS based on total dry matter. The HA-GOS preparation has a composition similar to a GOS preparation that is prepared from purified lactose feed. In addition, vitamins that were present in the CWP lactose feed may be present in the HA-GOS preparation. Also (part of) the minerals present in the SL-CWP lactose feed may be present.

It was observed that the HA-GOS obtained by the method of the present invention is richer in 4GL then conventional GOS. 4GL is a DP3 GOS with the formula galactose (ß1→4) lactose. 4GL is a human milk saccharide (HMO).

At the same time, it was observed that the HA-GOS obtained by the method of the present invention is rich in non-protein nitrogen (NPN). NPN originates from urea, ammonia, choline, amino sugars, uric acid, creatine/creatinine, carnitine, free amino acids, peptides, phospholipids, polyamines, and/or nucleotides/nucleosides. NPN is thought to play a role in gut maturation, microbiome development, satiety and satiation, adipogenesis, brain development and/or modulation of the immune system.

A further embodiment relates to a hypoallergenic GOS preparation of the invention for use in a method of at least partially preventing an (IgE-mediated) allergic response in a subject. Also provided is a method of at least partially preventing an (IgE-mediated) allergic response in a subject, comprising administering to the subject to a hypoallergenic GOS preparation of the invention. For example, the subject is known to suffer or has an increased chance to suffer from hypersensitivity to a GOS preparation obtained by transgalactosylation of lactose using a beta-galactosidase derived from Bacillus circulans.

In these embodiments, the subject is a mammal, in particular a human being. The subject may have any age. In a preferred embodiment, the subject is an adolescent or an adult. An adolescent is herein defined as a person having an age of from 13 to 20 years. An adult is herein defined as a person having an age of 20 years or higher. In another preferred embodiment, the subject is a child having an age of 3 years (36 months) to 13 years. In yet another preferred embodiment the subject is child having an age of 0 to 3 years, preferably having an age of 18 months or above, more preferably having an age of 24 months or above. The rare GOS-related allergy has thus far not been reported in subjects having an age of 18 months or below. Therefore, in a preferred embodiment, the subject is an adult, an adolescent, or a child, having an age of 18 months or above, preferably having an age of 24 months or above, more preferably having an age of 36 months or above.

In view of the localized incidence of the 4′-GOS and/or 6′-GOS-related allergies in South East Asia (e.g. Singapore, Japan), the subject is preferably of South East Asian origin.

### Hypoallergenic Nutritional Compositions

The invention also relates to a nutritional composition comprising the HA-GOS preparation according to the method of the invention for the production of such HA-GOS preparation. In one embodiment, the nutritional composition is a MUM composition for pregnant women, a growing up milk (GUM), a follow-up formula or an infant formula.

The invention also relates to a method for providing a hypoallergenic nutritional composition, comprising (i) providing the GOS preparation according to a method herein disclosed, and (ii) formulating said hypoallergenic GOS preparation together with at least one further hypoallergenic or non-allergenic ingredient into a hypoallergenic nutritional composition. The at least one further ingredient selected from the group consisting of a hypoallergenic or non-allergenic protein source may comprise a non-allergenic milk protein hydrolysate, free amino acids, probiotics, a lipid source, and carbohydrates, such as lactose, saccharose, starch or maltodextrin. The invention also provides a nutritional composition obtainable by such method.

Hypoallergenic or non-allergenic protein sources are known in the art, particularly for employment in infant formula. The terms non-allergenic hydrolysates and hydrolysates substantially free of allergenic proteins as used herein are interchangeable. They refer to protein hydrolysates that can be administered to infants having intolerance against dietary proteins, more particularly cow's milk proteins, without inducing allergic reactions. In one embodiment, the at least one further hypoallergenic or non-allergenic ingredient is selected from non-allergenic protein hydrolysates and hydrolysates substantially free of allergenic proteins, hypoallergenic protein sources, and hydrolyzed whey proteins. For example, U.S. Pat. No. 5,039,532 discloses a hydrolyzed whey protein material from which allergens consisting of alpha-lactalbumin, beta-lactoglobulin, serum albumin and immunoglobulins have not been removed and wherein the hydrolyzed protein material including hydrolyzed allergens is in a form of hydrolysis residues having a molecular weight not above 10,000 Da so that the hydrolyzed material is substantially free from allergenic proteins and allergens of protein origin. In one embodiment, a low-allergenic casein hydrolysate with peptides of maximally 3000 Da is included.

As a carbohydrate source, any type of carbohydrate, or a mixture of different carbohydrates, can serve which is normally used in children's food formulations. Suitable carbohydrate sources are disaccharides such as lactose and saccharose, monosaccharides, such as glucose, and maltodextrins, starch and carbohydrate sources having a prebiotic effect. In one embodiment, human milk oligosaccharides are used.

The lipid source in a composition according to the invention may be any type of lipid or combination of lipids which are suitable for use in (children's) nutritional products. Examples of suitable lipid sources are tri, di, and monoglycerides, phospholipids, sphingolipids, fatty acids, and esters or salts thereof. The lipids may have an animal, vegetable, microbial or synthetic origin. Of particular interest are polyunsaturated fatty acids (PUFAs) such as gamma linolenic acid (GLA), dihomo gamma linolenic acid (DHGLA), arachidonic acid (AA), stearidonic acid (SA), eicosapentaenoic acid (EPA), docosahexaenoic acid (DHA), docosapentaenoic acid (DPA) and conjugated linoleic acid (CLA). CLA is important in the protection against eczema and respiratory diseases in children. This particularly involves the cis-9, trans-11 and cis-12 isomers of CLA. Examples of suitable vegetable lipid sources include sun flower oil, high oleic sun flower oil, coconut oil, palm oil, palm kernel oil, soy bean oil, etc. Examples of suitable lipid sources of animal origin include milkfat, for example anhydrous milkfat (AMF), cream, etc. In a preferred embodiment, a combination of milkfat and lipids of vegetable origin are used.

In an embodiment, the composition according to the invention comprises a probiotic. In the context of the present invention, the term “probiotic” refers to a strain of probiotic bacteria. Probiotic bacteria are known in the art. Suitably, the probiotic bacteria are not genetically modified. Suitable probiotic bacteria include bacteria of the genus Bifidobacteria (e.g. B. breve, B. longum, B. infantis, B. bifidum), Lactobacillus (e.g. L. Acidophilus, L. paracasei, L. johnsonii, L. plantarum, L. reuteri, L. rhamnosus, L. casei, L. lactis), and Streptococcus (e.g. S. thermophilus). B. breve and B. longum are especially suitable probiotics. Suitable B. breve strains may for example be isolated from the faeces of healthy human milk-fed infants.

The combination of a prebiotic and a probiotic is also referred to as a “synbiotic”. The probiotic may be present in the composition at any suitable concentration, suitably in a therapeutically effective amount or “amount effective for treating” in the context of the invention. Suitably, the probiotic is included in the present composition in an amount of 10exp2-10exp13 cfu per g dry weight of the composition, suitably 10exp5-10exp12 cfu/g, most suitably 10exp7-10exp10 cfu/g.

Further, the composition may contain one or more conventional micro ingredients, such as vitamins, antioxidants, minerals, free amino acids, nucleotides, taurine, carnitine and polyamines. Examples of suitable antioxidants are BHT, ascorbyl palmitate, vitamin E, alpha and beta carotene, lutein, zeaxanthin, lycopene and phospholipids.

The composition according to the invention can be used as a nutritional composition, nutritional therapy, nutritional support, as a medical food, as a food for special medical purposes or as a nutritional supplement. The present composition is suitably an enteral composition. The composition is administered to, or intended to be administered to, a subject in need thereof. The subject is a mammal, in particular a human being, and the subject may have any age. In a preferred embodiment, the subject is an adult. The subject may e.g. be an elderly person or a post-menopausal woman. The subject may also be a pregnant woman. Thus, in some embodiments, the present composition is a MUM composition for pregnant women. In another preferred embodiment, the subject is an adolescent. In a specific aspect, the nutritional composition is a growing up milk (GUM) or a follow-up formula.

A nutritional composition provided herein, comprising HA-GOS obtained from a low-cost lactose feed, has many advantageous applications in the nutritional field. For example, the invention provides a method for at least partially preventing hypersensitivity to a GOS preparation in a subject, comprising administering a hypoallergenic nutritional composition comprising a hypoallergenic GOS preparation of the invention to the subject, preferably wherein the subject is an adult, an adolescent, or a child, more preferably having an age of 18 months or above. In a specific aspect, the subject is of South East Asian origin.

### LEGENDS TO THE FIGURES

FIG. 1: Illustration of OPL composition (in dry matter).

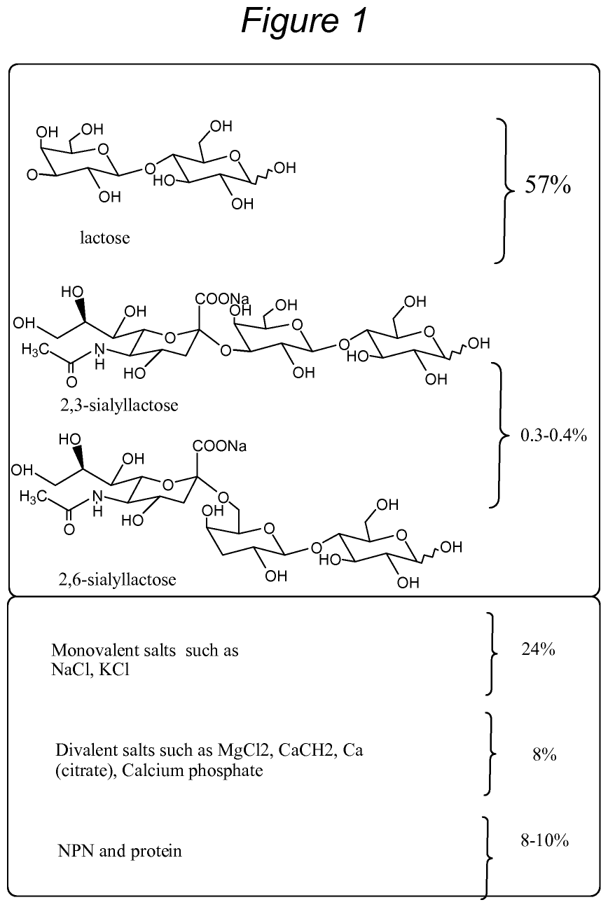

FIG. 2: Schematic illustration of enzymatically converting OPL into BOS.

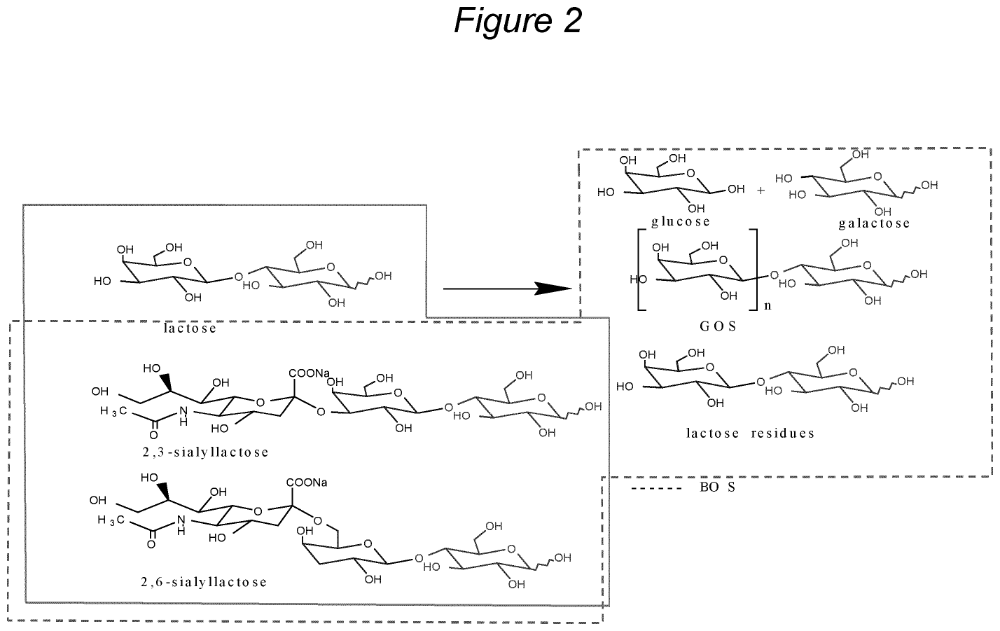

FIG. 3: Illustration of “OPL to SL-enriched GOS” process.

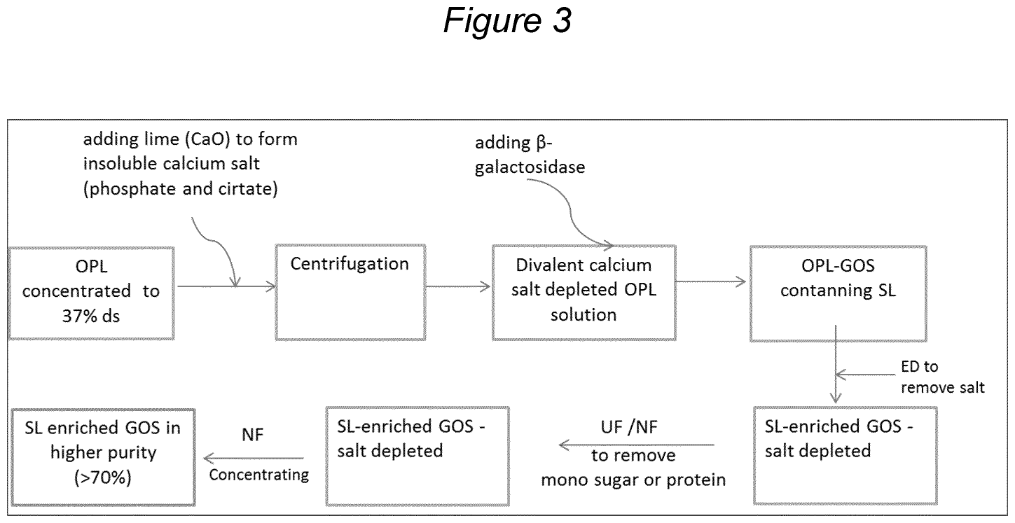

FIG. 4A: Results of the Basophil activation in test subject #1 as measured by expression of the basophil activation marker CD203c (MFl=Mean Fluorescence).

FIG. 4B: Results of the Basophil activation in test subject #2 as measured by expression of the basophil activation marker CD203c (MFl=Mean Fluorescence).

FIG. 4C: Results of the Basophil activation in test subject #3 as measured by expression of the basophil activation marker CD203c (MFl=Mean Fluorescence).

### EXPERIMENTAL SECTION

Beta-Galactosidase Derived from C. terrestris

The beta-galactosidase enzymes according to SEQ ID NO: 1, 2, 3 and 4 derived from C. terrestris (also referred to as “Tetris enzyme”) for use in the present invention were obtained from Amano Enzyme Inc. (Nagoya, Japan). These beta-galactosidase enzymes and methods for their preparation are disclosed in WO 2017/115826 by Amano. Methods for the preparation of (mutant) beta-galactosidase enzymes derived from C. terrestris, as well as the enzyme properties, are disclosed in WO 2017/115826.

### Example 1: Preparation of Hypoallergenic GOS (HA-GOS)

This example describes the process of determining the optimal process conditions for the Tetris enzyme to prepare HA-GOS from Cheese Whey Permeate (CWP) as lactose feed.

Electrodialysis (ED) was used to demineralize the Cheese Whey Permeate from about 12 mS to 4 mS or below. After concentration, the demineralized CWP was used as lactose feed in the preparation of HA-GOS.

The composition of the cheese whey permeate before and after demineralization is shown in Table 3.

TABLE 3
Composition of cheese whey permeate (CWP).

| | CWP (12.7 mS) | Demineralized CWP (4 mS) |
|---|---|---|
| Dry matter % | 28.0 | 26.8 |
| Total Protein % | 0.77 | 0.73 |
| NPN % | 0.70 | 0.64 |
| Lactose % | 24.3 | 24.7 |
| Ash % | 2.52 | 0.93 |
| Potassium % | 0.78 | 0.188 |
| Sodium % | 0.193 | 0.101 |
| Magnesium mg/kg | 372 | 270 |
| Calcium mg/mg | 1500 | 1200 |
| Phosphorous % | 0.167 | 0.125 |
| Chloride % | 0.66 | 0.075 |
| Nitrate mg/kg | 334 | 116 |
| Nitrite mg/kg | <0.1 | <0.1 |
| Ash on DM % | 9.00 | 3.5 |
| Lactose on DM % | 86.8 | 92.2 |
| Protein on DM % | 2.75 | 2.72 |

### Experimental Set-Up

### Temperature: 65° C., 67.5° C. or 70° C.

Lactose content (DM): 45, 50 or 55%
Enzyme dosage: 50%, 75% or 100%, wherein 100%=0.94 LU/gram lactose

Cheese whey permeate (CWP) was used as lactose feed. The CWP was first demineralized to 4% mineral content. This demineralized CWP was then concentrated (via evaporation) to a lactose content of either 45%, 50% or 55%. Then the GOS reaction was initiated by adding different dosages (0.5, 0.75 or 1.0) of the Tetris enzyme (batch BGP1150931SDR). The GOS reaction took place in a water bath using magnetic stirring, in the dark. No pH adjustments before or during the process were performed. The reaction temperature was either 65, 67.5 or 70° C.

The experimental details of the various experiments are summarized in Table 4.

TABLE 4
Details of various experiments to prepare
HA-GOS from demineralized CWP.

| | | | |
|---|---|---|---|
| | | Lactose concentration | Enzyme |
| Temp. | Exp. Nr. | (% DM) | dosage |

| | | | | |
|---|---|---|---|---|
| 65° | C. | 1 | 45 | 0.5 |
| | | 2 * | 50 | 0.75 |
| | | 3 | 55 | 1 |
| | | 10 | 45 | 0.75 |
| 67.5° | C. | 13 | 45 | 0.75 |
| | | 14 | 50 | 1 |
| | | 15 | 55 | 0.5 |
| | | 22 | 45 | 0.75 |
| 70° | C. | 25 | 45 | 1 |
| | | 26 | 50 | 0.5 |
| | | 27 | 55 | 0.75 |
| | | 34 | 45 | 0.75 |

* this sample was used in the BAT test to determine hypoallergenicity.

### GOS Yield

Tables 5, 6 and 7 show the amount of GOS, based on lactose, in several experiments at 65° C. (Table 5), 67.5° C. (Table 6) and 70° C. (Table 7). The results indicate how effective the enzyme is in converting lactose to GOS at various reactions conditions. Also it shows the amount of GOS based on dry matter (DM) and based on lactose. A third column is added with the amount of GOS based on total product. The highest yield is possible with the highest DM. It also shows that 0.5 of the normal dosage of enzyme is sufficient to make more than 45% GOS on DM even after 24 hours.

TABLE 5
GOS concentrations of GOS, based on lactose, on DM and
on total product for a temperature of 65° C., at
various lactose concentrations, using CWP as the lactose feed.

| Reaction | | Enzyme Dosage | | GOS on | GOS on | GOS on |
|---|---|---|---|---|---|---|
| time (h) | lactose | (LU/gram lactose) | total DM | Lactose | DM | product |
| 24 | 45 | 0.50 | 49.09 | 54.20 | 49.86 | 24.48 |
| 48 | 45 | 0.50 | 49.09 | 58.53 | 53.85 | 26.44 |
| 24 | 50 | 0.75 | 54.55 | 57.71 | 53.09 | 28.96 |
| 48 | 50 | 0.75 | 54.55 | 61.42 | 56.51 | 30.82 |
| 24 | 55 | 1.00 | 59.34 | 59.21 | 54.47 | 32.32 |
| 48 | 55 | 1.00 | 59.34 | 62.07 | 57.10 | 33.89 |

TABLE 6
GOS concentrations of GOS, based on lactose, on DM and on
total product for a temperature of 67.5° C., at various
lactose concentrations, using CWP as the lactose feed.

| Reaction | | Enzyme Dosage | | GOS on | GOS on | GOS on |
|---|---|---|---|---|---|---|
| time (h) | lactose | (LU/gram lactose) | total DM | Lactose | DM | product |
| 24 | 45 | 0.75 | 49.09 | 52.35 | 48.16 | 23.64 |
| 48 | 45 | 0.75 | 49.09 | 60.95 | 56.07 | 27.53 |
| 24 | 50 | 1 | 54.55 | 57.19 | 52.61 | 28.70 |
| 48 | 50 | 1 | 54.55 | 62.06 | 57.10 | 31.14 |
| 24 | 55 | 0.5 | 59.67 | 53.32 | 49.05 | 29.27 |
| 48 | 55 | 0.5 | 59.67 | 58.59 | 53.90 | 32.16 |

TABLE 7
GOS concentrations of GOS, based on lactose, on DM and
on total product for a temperature of 70° C., at
various lactose concentrations, using CWP as the lactose feed.

| Reaction | | Enzyme Dosage | | GOS on | GOS on | GOS on |
|---|---|---|---|---|---|---|
| time (h) | lactose | (LU/gram lactose) | total DM | Lactose | DM | product |
| 24 | 45 | 1 | 49.09 | 60.1 | 55.29 | 27.14 |
| 48 | 45 | 1 | 49.09 | 60.62 | 55.77 | 27.38 |
| 24 | 50 | 0.5 | 54.55 | 55.51 | 51.07 | 27.86 |
| 48 | 50 | 0.5 | 54.55 | 58.74 | 54.04 | 29.48 |
| 24 | 55 | 0.75 | 60.00 | 59.08 | 54.35 | 32.61 |
| 48 | 55 | 0.75 | 60.00 | 61.34 | 56.43 | 33.86 |

Tables 8 and 9 show the average values for the HA-GOS yield for the different parameters that were varied in the series of experiments.

TABLE 8
Average values of GOS yield, expressed as GOS based on
lactose content (meaning that x % of all lactose in sample
is converted into GOS), after 24 h reaction time.

| Reaction time 24 h | | GOS, based on lactose (%) |
|---|---|---|
| Temperature effect | 65° C. | 57.2 |
| | 67.5° C. | 55.7 |
| | 70° C. | 58.3 |
| Enzyme dosage | 0.5 | 54.5 |
| | 0.75 | 57.2 |
| | 1 | 59.5 |
| Lactose content | 45% | 56.6 |
| | 50% | 57.3 |
| | 55% | 57.3 |

TABLE 9
Average values of GOS yield, expressed as GOS based on
lactose content (meaning that x % of all lactose in sample
is converted into GOS), after 24 h reaction time.

| Reaction time 42 h | | GOS, based on lactose (%) |
|---|---|---|
| Temperature effect | 65° C. | 60.7 |
| | 67.5° C. | 58.4 |
| | 70° C. | 60.8 |
| Enzyme dosage | 0.5 | 56.7 |
| | 0.75 | 61.4 |
| | 1 | 61.7 |
| Lactose content | 45% | 60.6 |
| | 50% | 60.6 |
| | 55% | 58.6 |

Further characteristics of a HA-GOS preparation according to the invention are shown in Tables 10 and 11. Table 10 shows the sugar profile of HA-GOS according to the invention compared to that of a reference GOS preparation obtained with purified lactose as lactose feed using the Tetris enzyme.

TABLE 10
Sugar profile of HA-GOS according to the invention and of a reference GOS.

| | | | Ref. GOS ¹ | HA-GOS ² |
|---|---|---|---|---|
| DM | % | | | 51.67 |
| Lactose | % | | | 46.50 |
| Conc. on Brix | % based on lactose | galactose | 1.25 | 0.93 |
| Conc. on Brix | % based on lactose | glucose | 19.57 | 17.97 |
| Conc. on Brix | % based on lactose | allolactose | 3.24 | 0.77 |
| Conc. on Brix | % based on lactose | lactose | 15.80 | 20.40 |
| Conc. on Brix | % based on lactose | lactulose | 0.57 | 0.59 |
| % GOS | % based on lactose | | 59.6 | 59.3 |
| % GOS | % based on DM | | 59.7 | 53.4 |

¹ Ref. GOS is a reference GOS preparation obtained with purified lactose as lactose feed using the Tetris enzyme.
² HA-GOS preparation according to the invention, obtained from CWP using the Tetris enzyme.

Table 11 shows the distribution of di-to heptasaccharides (degree of polymerization (DP) 2 to 7) of HA-GOS according to the invention compared to that of a reference GOS obtained with purified lactose as lactose feed using the Tetris enzyme.

TABLE 11
DP distribution of HA-GOS according to the invention
compared to that of a reference GOS.

| | Ref. GOS ¹ | HA-GOS ² |
|---|---|---|
| Galactose | 1.7 | 0.7 |
| Glucose | 19.4 | 18.5 |
| DP2 | 39.7 | 43.6 |
| DP3 | 25.5 | 27.8 |
| DP4 | 11.7 | 8.7 |
| DP5 | 1.9 | 0.7 |
| DP6 | 0.2 | 0.0 |
| DP7 | 0.0 | 0.0 |
| Total | 100.0 | 100.0 |

¹ Ref. GOS is a reference GOS preparation obtained with purified lactose as lactose feed using the Tetris enzyme.
² HA-GOS preparation according to the invention, obtained from CWP using the Tetris enzyme.

From the above it follows that when CWP is used as lactose feed in a method for the production of a HA-GOS preparation, excellent results are obtained when the minimal enzyme dosage is 0.65 LU/gram lactose, the minimal lactose concentration is >50% at a temperature of 70° C. for a 24 hour reaction time, when CWP is used as raw material. With a reaction time of 42 hours the optimal reaction conditions are an enzyme dosage of 0.75 LU/gram, a temperature of 65° C. and a lactose concentration of 50%.

When selecting the optimal conditions it is important to bear it in mind that the best conditions are in a certain range and the specific conditions can be defined when combining all 3 selected parameters in the optimal range. For instance, when selecting 0.75 LU/grams the enzyme dosage, the temperature is 65° C. and the lactose concentration is ≥50%.

### Example 2: Preparation of HA-GOS from SL-CWP

This example describes how CWP enriched in SL (SL-CWP) can be obtained, and used as lactose feed for the cost-effective manufacture of HA-GOS.

CWP was first subjected to a lactose removal step by crystallization using procedures known in the art. To this end, CWP is concentrated typically to ˜48% (w/w) and the solution is cooled down to 15° C. and is stirred for 8-24 hour to allow for lactose crystallization. The so formed lactose crystals are separated by centrifugation. The mother liquor obtained is called OPL or DLP and typically contains about 24% (w/w) dry matter. OPL or DLP is concentrated to 44% (w/w) or higher for further application or treatment. The OPL or DLP is enriched in sialyllactose SL and can be subjected to various treatments mentioned above to obtain a HA-GOS enriched in SL with high purity (>75% GOS) after removal of salts and mono sugars.

The composition of the SL-CWP thus obtained is shown in Table 12.

TABLE 12
Typical chemical composition of SL-CWP
(lactose-depleted whey; OPL) and CWP.

| | SL-CWP | CWP |
|---|---|---|
| Dry matter content (%) | 24 | 27 |
| SA (sialic acid) (%) | 0.11 | 0.03 |
| 3′-SL (%) | 0.22 | 0.03 |
| 6′-SL (%) | 0.08 | 0.06 |
| SL total (%) | 0.30 | 0.1 |
| Lactose (%) | 57 | 95 |
| Protein | 7.8 | |
| NPN (%) | 9 | 1.5 |
| Ash (%) | 23.7 | |

HA-GOS Preparation Using the Tetris Enzyme, with SL-CWP as Lactose Feed.

The reaction was performed at 65° C. using 65% (w/w) SL-CWP (prepared as described above) with an enzyme dosage of 2 LU/gram lactose. After 24 hour reaction time the formed HA-GOS was separated by centrifugation and the enzyme was denatured by heating at 100° C. for 15 minutes. The final product was analyzed by HPLC to determine the DP (degree of polymerization) distribution. It was found that the DP composition is highly similar to a reference GOS that was prepared with the Tetris beta-galactosidase using purified lactose as lactose feed (see Table 13). Following the removal of minerals, the GOS level is at least about 45 wt. % and the SL level is at least about 0.4 wt. %.

TABLE 13
Comparison between the DP composition of a conventional
GOS preparation produced with Bacillus circulans beta-
galactosidase (BC-GOS), a reference GOS obtained from purified
lactose using “Tetris” enzyme derived from Cryptococcus terrestris
(Ref-GOS) and HA-GOS obtained from SL-CWP using “Tetris”
enzyme derived from Cryptococcus terrestris according
to the invention.

| DP | BC-GOS ¹ | Ref-GOS ² | HA-GOS ³ |
|---|---|---|---|
| DP7 | 0.53 | 0 | 0 |
| DP6 | 1.8 | 0.19 | 0.38 |
| DP5 | 4.79 | 1.98 | 2.08 |
| DP4 | 10.74 | 12.06 | 11.15 |
| DP3 | 22.02 | 25.47 | 24.28 |
| DP2 | 37.83 | 39.06 | 39.06 |
| Glucose | 20.5 | 18.68 | 21.21 |
| Galactose | 1.8 | 2.55 | 1.85 |

¹ BC-GOS is a conventional GOS preparation produced with purified lactose as lactose feed and with a Bacillus circulans beta-galactosidase.
² Ref. GOS is a reference GOS preparation obtained with purified lactose as lactose feed using the Tetris enzyme.
³ HA-GOS preparation according to the invention, obtained from SL-CWP using the Tetris enzyme.

Tables 14 and 15 summarize the composition of HA-GOS obtained using either purified lactose, CWP or SL-CWP as a lactose feed in a transgalactosylation method employing “Tetris” enzyme. Also included are BC-GOS, referring to a commercial GOS preparation obtained using B. circulans beta-galactosidase, and the composition of the crude starting materials CWP and SL-CWP.

TABLE 14
Composition of GOS obtained using purified lactose as a lactose
feed in a transgalactosylation method employing “Tetris”
enzyme (Ref-GOS) or B. circulans beta-galactosidase (BC-GOS).

| | | | Ref-GOS² | Ref-GOS² | Ref-GOS² | Ref-GOS² |
|---|---|---|---|---|---|---|
| | | BC-GOS¹ | PT731 | PT741 | PT631 | PT431 |
| GOS | % DM | 58.14 | 62.2 | 63.14 | 63.8 | 64.82 |
| | % lactose | | | | | |
| glucose | % on DM | 20.36 | 18.4 | 18.16 | 18.39 | 17.24 |
| lactose | % on DM | 20.04 | 18.3 | 17.6 | 16.62 | 17.07 |
| galactose | % lactose | 1.46 | 1.03 | 1.1 | 1.19 | 0.88 |
| sialic acid | % on DM | | | | | |
| 3′SL | % on DM | | | | | |
| 6′SL | % on DM | | | | | |
| SL | % on DM | | | | | |
| DM | % | 74.26 | 76.08 | 75.09 | 76.39 | 76.79 |
| protein | on DM | 0.01 | 0.01 | <0.01 | <0.01 | <0.01 |
| NPN | on DM | 0.0016 | 0.0016 | <0.0016 | <0.0016 | <0.0016 |
| nitrite | on DM | 0.02 | 0.08 | 0.07 | 0.07 | 0.07 |
| ash (525C) | on DM | 0.1 | <0.01 | 0.02 | 0.02 | 0.02 |
| calcium | on DM | | | | | |
| magnesium | on DM | | | | | |
| potassium | on DM | | | | | |
| | % phosphate | | | | | |
| sodium | on DM | | | | | |
| phosphate inorganic | % w/w | | | | | |
| pH | | 2.9 | 3.34 | 3.24 | 3.08 | 3.2 |
| GOS DP | | | | | | |
| DP2 (other than lactose) | | 37.4 | 41.15 | 41.21 | 40.59 | 41.25 |
| DP3 | % GOS cont. | 22.02 | 26.02 | 25.99 | 25.63 | 26.43 |
| DP4 | % GOS cont. | 10.76 | 11.3 | 11.26 | 11.67 | 11.67 |
| DP5 | % GOS cont. | 4.88 | 1.56 | 1.53 | 1.71 | 1.6 |
| DP6 | % GOS cont. | 1.9 | 0.14 | 0.14 | 0.16 | 0.15 |
| DP7 | % GOS cont. | 0.6 | — | — | — | — |

¹BC-GOS is a conventional GOS preparation produced with purified lactose as lactose feed and with a Bacillus circulans beta-galactosidase.
²Ref. GOS is a reference GOS preparation obtained with purified lactose as lactose feed using the Tetris enzyme.

TABLE 15
Composition of HA-GOS obtained using either CWP or SL-CWP as a lactose
feed in a transgalactosylation method employing “Tetris” enzyme,
and the composition of the crude starting materials CWP and SL-CWP.

| | | | HA-GOS | SL-CWP | HA-GOS |
|---|---|---|---|---|---|
| | | CWP | (CWP) 1 | (OPL) | (SL-CWP) 2 |
| GOS | % DM | | 53.4 | | ca. 55* |
| | % lactose | | 59.3 | | |
| glucose | % on DM | | 18.0 | — | 21.21 |
| lactose | % on DM | 83 | 20.4 | 57.0 | ca. 20* |
| galactose | % lactose | | 0.9 | | 1.85 |
| sialic acid | % on DM | 0.03 | | 0.1 | 0.11 |
| 3′SL | % on DM | 0.03 | | 0.2 | 0.22 |
| 6′SL | % on DM | 0.06 | | 0.1 | 0.08 |
| SL total | % on DM | 0.1 | | 0.3 | 0.3 |
| DM | % | 27.6 | 70.6 | 24.0 | 24 |
| protein | on DM | 2.7 | 2.6 | 7.8 | 7.8 |
| NPN | on DM | 0.4 | 0.4 | 9.0 | 9 |
| nitrite | on DM | | <0.1 | | |
| ash (525C) | on DM | 9.02 | 3.3 | 23.7 | 23.7 |
| calcium | on DM | | 4166.7 | | |
| magnesium | on DM | | 1017.4 | | |
| potassium | on DM | | 7013.9 | | |
| | % phosphate | | | | |
| sodium | on DM | | 2951.4 | | |
| phosphate inorganic | % w/w | | 0.4 | | |
| pH | | | 5.1 | | |
| GOS DP | | | | | |
| DP2 (other than lactose) | | | 43.6 | | 39.06 |
| DP3 | % GOS cont. | | 27.8 | | 24.28 |
| DP4 | % GOS cont. | | 8.7 | | 11.15 |
| DP5 | % GOS cont. | | 0.7 | | 2.08 |
| DP6 | % GOS cont. | | — | | 0.38 |
| DP7 | % GOS cont. | | — | | — |

1 HA-GOS (CWP) preparation according to the invention, obtained from CWP using the Tetris enzyme.
2 HA-GOS (SL-CWP) preparation according to the invention, obtained from SL-CWP using the Tetris enzyme.

### Example 3: Basophil Activation Test

This example describes a basophil activation test to demonstrate the reduced allergenicity of HA-GOS in multiple human subjects with known galacto-oligosaccharide allergy. The test was performed with HA-GOS prepared with cheese whey permeate (CWP) as lactose feed and with HA-GOS prepared with cheese whey permeate enriched in SL (SL-CWP) as lactose feed. HA-GOS prepared with CWP as lactose feed is herein referred to as HA-GOS (CWP). HA-GOS prepared with SL-CWP as lactose feed is herein referred to as HA-GOS (SL-CWP).

### Materials

HA-GOS (CWL) (batch PT731; see Example 1), HA-GOS (SL-CWP) (see Example 2) and a commercial GOS preparation obtained using B. circulans enzyme (BC-GOS) were included in the tests. The materials were stored at room temperature in the dark until use.

### Subjects

Eligible subjects were selected from the cohort previously studied for the prevalence of GOS-allergy in a Singapore atopic population, as described in the paper by Soh et al. (Anaphylaxis to galacto-oligosaccharides—an evaluation in an atopic population in Singapore, Allergy, 2015, 70, 1020-1023). The study was approved by the National University Hospital Singapore institutional ethical review board. Written consent of all subjects was obtained prior to the start of the study.

### Basophil Activation Test

A Basophil Activation Test was performed on patient blood samples. Heparinized peripheral blood aliquots (100 μL) were pre-incubated at 37° C. for 5 minutes and then incubated with 100 μL of PBS (negative control), anti-IgE antibody (positive control, G7-18; BD Biosciences, San Jose, Calif.) or diluted GOS samples for 15 minutes (37° C.). After incubation, cells were washed in PBS-EDTA (20 mmol/L) and then incubated with phycoerythrin-labeled anti-human IgE (Ige21; eBioscience, San Jose, Calif.), biotin-labeled anti-human CD203c (NP4D6; BioLegend, San Jose, Calif.), and fluorescein isothiocyanate-labeled anti-human CD63 (MEM-259, BioLegend) mAbs for 20 minutes at 48° C. Expression of CD203c and CD63 are both markers for basophil activation. After washing the cells with 1% BSA/PBS, allophycocyanin-conjugated streptavidin (BD Biosciences) was added and incubated for 15 minutes at 48° C. Thereafter, samples were subjected to erythrocyte lysis with 2 mL of FACS Lysing Solution (BD Biosciences). Cells were then washed, resuspended in 1% BSA/PBS, and analysed by means of FACSCalibur (BD Biosciences). Basophils were detected on the basis of side-scatter characteristics and expression of IgE (IgEhigh).

### Results

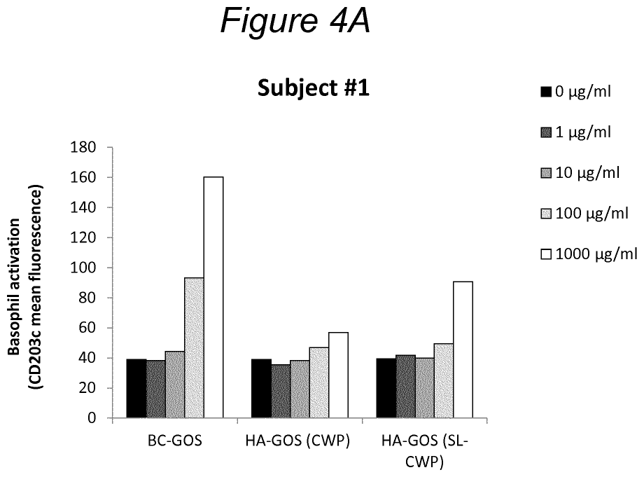

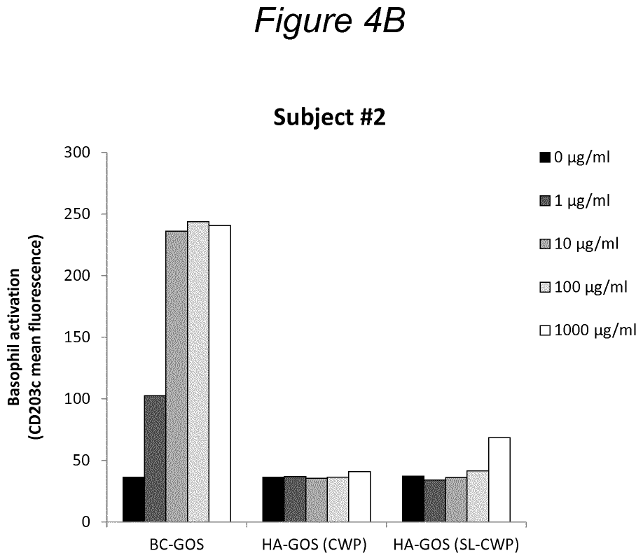

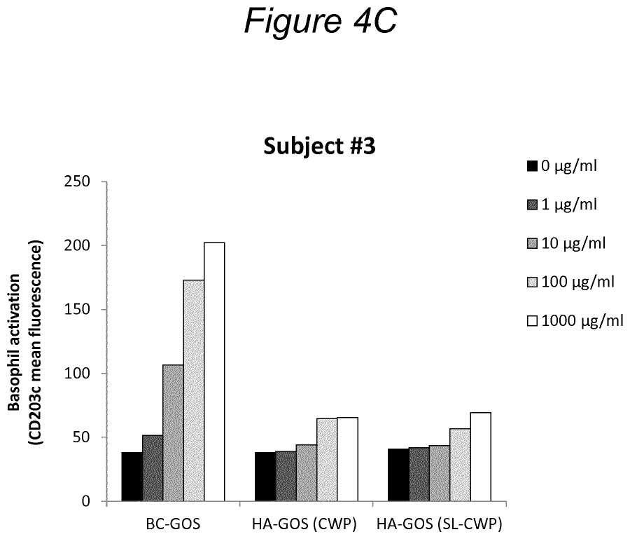

FIGS. 4a-4c show the results of the basophil activation test performed on three subjects. FIG. 4a shows the results of the Basophil activation in test subject #1 as measured by expression of the basophil activation marker CD203c (MFl=Mean Fluorescence). Similarly, FIGS. 4b and 4c show the Basophil activation in test subjects #2, and #3, respectively,

The results show that all three subjects had a positive basophil activation response to BC-GOS, which indicates that the subjects are allergic to BC-GOS and thus confirms previous findings (Soh et al., 2015). However, incubation of basophils with HA-GOS (CWP) or HA-GOS (SL-CWP) showed no or hardly any basophil activation in any of the subjects. Only at the highest concentrations tested, HA-GOS (CWP) and HA-GOS (SL-CWP) showed very slight basophil activation, which was clearly decreased as compared to the reference BC-GOS response.

### CONCLUSIONS

Basophil activation tests in BC-GOS allergic subjects showed that HA-GOS (CWP) and HA-GOS (SL-CWP) have a markedly reduced allergenicity as compared to BC-GOS. It can be concluded that HA-GOS (CWP) and HA-GOS (SL-CWP) according to the invention are clearly hypoallergenic.

1: A method of producing a hypoallergenic galacto-oligosaccharide (HA-GOS) preparation, the method comprising contacting a lactose feed with a beta-galactosidase (EC 3.2.1.23) comprising an amino acid sequence according to any of SEQ ID NO: 1, 2, 3 or 4, or an amino acid sequence that is at least 80% identical thereto, wherein the lactose feed comprises a cheese whey permeate (CWP) or a CWP that is enriched in sialyllactose (SL-CWP).

2: The method according to claim 1, wherein the lactose feed comprises a demineralized CWP, preferably CWP that has been demineralized, more preferably by electrodialysis (ED), to an ash content of up to 4 wt. % and/or a conductivity of up to 4 mS.

3: The method according to claim 1, wherein the lactose feed is contacted with beta-galactosidase at a temperature of about 65-70° C., at a lactose content in the range of 47-55 wt. % lactose, and/or an enzyme dosage of 0.75-1.0 LU/gram.

4: The method according to claim 1, wherein the lactose feed comprises a SL-CWP, preferably having sialyllactose content of at least 0.25 wt. %, more preferably at least 0.3 wt. % based on dry solids.

5: The method according to claim 4, wherein the SL-CWP is a CWP that has been subjected to a lactose-removal step, preferably followed by a step to remove multivalent anions.

6: The method according to claim 4, wherein the lactose-content of the SL-CWP is about 52-60 wt. %.

7: The method according to claim 4, wherein the lactose feed is contacted with beta-galactosidase at a temperature of about 55-60° C., at pH 6.0-7.0 and/or at an enzyme dosage of 0.75-1.0 LU/gram.

8: The method according to claim 1, wherein the HA-GOS thus obtained is subjected to at least one further purification step, preferably wherein said purification step comprises one or more of demineralization, de-proteinization, removal of mono-sugar components, enriching for sialyllactose and removal of coloured impurities.

9: The method according to claim 1, wherein the beta-galactosidase comprises an amino acid sequence that is at least 85%, preferably at least 90%, more preferably at least 95%, yet even more preferably at least 97%, and most preferably at least 98% identical to any of SEQ ID NO: 1, 2, 3 or 4.

10: The method according to claim 1, wherein the beta-galactosidase (EC 3.2.1.23) is derived from Cryptococcus terrestris strain MM13-F2171 (Accession Number: NITE BP-02177) or APC-6431 (Accession Number: NITE BP-02178).

11: A hypoallergenic GOS preparation obtained by the method according to claim 1.

12: The hypoallergenic GOS preparation according to claim 11, showing a decreased score in a Basophil Activation Test performed on a blood sample isolated from a subject when compared to a GOS preparation obtained by transgalactosylation of lactose using Bacillus circulans beta-galactosidase.

13: A method of at least partially preventing an (IgE-mediated) allergic response in a subject known to suffer or has an increased chance to suffer from hypersensitivity to a GOS preparation obtained by transgalactosylation of lactose using a beta-galactosidase derived from Bacillus circulans, the method comprising administering to the subject an effective amount of the hypoallergenic GOS preparation of claim 11.

14: A nutritional composition comprising the hypoallergenic GOS preparation according to claim 11.

15: The nutritional composition according to claim 14, wherein the nutritional composition is a MUM composition for pregnant women, a growing up milk (GUM), a follow-up formula, or an infant formula.


SEQUENCE LISTING (4 sequences)

SEQ ID NO: 1 - protein, 696 aa, Cryptococcus terrestris
MIPASALLAAVPLLAQQVSAGILRRQNAAGSDSAAPDSIADASTGVVSSIATEAVSSGATGLVASVAMSFASSMATPTATVTGLSSETGAPSNTPMASASGSVPTTTSAVGSGDFDWVQTDGLPTITTTLATTNQDAITPTATGPVGGQGTPAVNFTDYSSSSLEQFWNDWVGEVEEPPFAYVPEPPNPYPLPNAPPPIYPEYYTKRPKDILPDYKFPKDFLFGWATAAQQWEGAVKADGKGPSIWDWASRFPGFIADNTTSDVGDLGYYLYKEDLARIAALGANVYSFSMFWTRIFPFGKADSPVNQAGIDFYHDLIDYSWSLGIEPVVTLFHWDTPLALQLEYGGFASERIIDDYVNYAETVFKAYNGSVHKWVTFNEPVVFCSQMAAPVNTTLPPNLNSTIYPYTCSYHLVLAHAKTVKRFRELNIQGQIAFKSDNFVGIPWREGNQEDIDAVERHQAYQIGIFAEPIYNTGDWPDIVKNDLSPDILPRFTDDEIAMIKCTADFFPIDGYRDGYVQAVPGGVEACVANISNPLWPACNQVNFYDSTPAGWAIGTFGNWPTTPWLQNTWQFVRPFLADLAKRYPTEGGIYLSEFGFSEPFENDKTFIYQITQDSGRTAYFNSYLGEVLKGIVEDGIPIKGVFGWSMVDNFEWNSGLSTRFGVQYVDYNSPTRQRTFKRSALEMSEFWNAHRCSA

SEQ ID NO: 2 - protein, 566 aa, Cryptococcus terrestris
ATTNQDAITPTATGPVGGQGTPAVNFTDYSSSSLEQFWNDWVGEVEEPPFAYVPEPPNPYPLPNAPPPIYPEYYTKRPKDILPDYKFPKDFLFGWATAAQQWEGAVKADGKGPSIWDWASRFPGFIADNTTSDVGDLGYYLYKEDLARIAALGANVYSFSMFWTRIFPFGKADSPVNQAGIDFYHDLIDYSWSLGIEPVVTLFHWDTPLALQLEYGGFASERIIDDYVNYAETVFKAYNGSVHKWVTFNEPVVFCSQMAAPVNTTLPPNLNSTIYPYTCSYHLVLAHAKTVKRFRELNIQGQIAFKSDNFVGIPWREGNQEDIDAVERHQAYQIGIFAEPIYNTGDWPDIVKNDLSPDILPRFTDDEIAMIKCTADFFPIDGYRDGYVQAVPGGVEACVANISNPLWPACNQVNFYDSTPAGWAIGTFGNWPTTPWLQNTWQFVRPFLADLAKRYPTEGGIYLSEFGFSEPFENDKTFIYQITQDSGRTAYFNSYLGEVLKGIVEDGIPIKGVFGWSMVDNFEWNSGLSTRFGVQYVDYNSPTRQRTFKRSALEMSEFWNAHRCSA

SEQ ID NO: 3 - protein, 560 aa, Cryptococcus terrestris
AITPTATGPVGGQGTPAVNFTDYSSSSLEQFWNDWVGEVEEPPFAYVPEPPNPYPLPNAPPPIYPEYYTKRPKDILPDYKFPKDFLFGWATAAQQWEGAVKADGKGPSIWDWASRFPGFIADNTTSDVGDLGYYLYKEDLARIAALGANVYSFSMFWTRIFPFGKADSPVNQAGIDFYHDLIDYSWSLGIEPVVTLFHWDTPLALQLEYGGFASERIIDDYVNYAETVFKAYNGSVHKWVTFNEPVVFCSQMAAPVNTTLPPNLNSTIYPYTCSYHLVLAHAKTVKRFRELNIQGQIAFKSDNFVGIPWREGNQEDIDAVERHQAYQIGIFAEPIYNTGDWPDIVKNDLSPDILPRFTDDEIAMIKCTADFFPIDGYRDGYVQAVPGGVEACVANISNPLWPACNQVNFYDSTPAGWAIGTFGNWPTTPWLQNTWQFVRPFLADLAKRYPTEGGIYLSEFGFSEPFENDKTFIYQITQDSGRTAYFNSYLGEVLKGIVEDGIPIKGVFGWSMVDNFEWNSGLSTRFGVQYVDYNSPTRQRTFKRSALEMSEFWNAHRCSA

SEQ ID NO: 4 - protein, 555 aa, Cryptococcus terrestris
ATGPVGGQGTPAVNFTDYSSSSLEQFWNDWVGEVEEPPFAYVPEPPNPYPLPNAPPPIYPEYYTKRPKDILPDYKFPKDFLFGWATAAQQWEGAVKADGKGPSIWDWASRFPGFIADNTTSDVGDLGYYLYKEDLARIAALGANVYSFSMFWTRIFPFGKADSPVNQAGIDFYHDLIDYSWSLGIEPVVTLFHWDTPLALQLEYGGFASERIIDDYVNYAETVFKAYNGSVHKWVTFNEPVVFCSQMAAPVNTTLPPNLNSTIYPYTCSYHLVLAHAKTVKRFRELNIQGQIAFKSDNFVGIPWREGNQEDIDAVERHQAYQIGIFAEPIYNTGDWPDIVKNDLSPDILPRFTDDEIAMIKCTADFFPIDGYRDGYVQAVPGGVEACVANISNPLWPACNQVNFYDSTPAGWAIGTFGNWPTTPWLQNTWQFVRPFLADLAKRYPTEGGIYLSEFGFSEPFENDKTFIYQITQDSGRTAYFNSYLGEVLKGIVEDGIPIKGVFGWSMVDNFEWNSGLSTRFGVQYVDYNSPTRQRTFKRSALEMSEFWNAHRCSA